(19) United States
(12) Patent Application Publication
(10) Pub. No.: US 2015/0320407 A1
(43) Pub. Date: Nov. 12, 2015

(54) SAMPLING APPARATUS AND METHOD

(71) Applicant: THE STATE OF QUEENSLAND THROUGH THE DEPARTMENT OF HEALTH, Brisbane, Queensland (AU)

(72) Inventor: Gerald Holtmann, Tennyson (AU)

(73) Assignee: The State of Queensland Through the Department of Health, Brisbane, Queensland (AU)

(21) Appl. No.: 14/402,308

(22) PCT Filed: May 17, 2013
(86) PCT No.: PCT/AU2013/000513

§ 371 Date: Nov. 19, 2014

(30) Foreign Application Priority Data
May 21, 2012 (AU) 2012902083

(51) Int. Cl. A61B 10/06; A61B 10/02; A61B 10/04

(52) U.S. Cl. CPC A61B 10/06; A61B 10/04; A61B 10/0233

(57) ABSTRACT

A sampling apparatus for extracting a sample from a biological subject, the apparatus including, a sampling device including a sampling tool for extracting a sample from the biological subject through a working channel of an endoscope and an isolating member for isolating at least the sample from the working channel during extraction of the sample.

### BACKGROUND OF THE INVENTION

The present invention relates to a method and apparatus for extracting a sample from a biological subject, and in one particular example, to an apparatus and method for extracting a sample from a subject as part of an endoscopic procedure.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

In Australia functional dyspepsia (FD) affects 15% of the population, causing considerable morbidity and is a common reason for medical consultations and a substantial financial burden. Whilst it is accepted that treatment with proton pump inhibitors, prokinetics, diets, psychotropic drugs and some herbal remedies may improve symptoms in a subset of patients, there is no cure for this disease and at best the gain over placebo reaches 20%. Thus there is a pressing need to unravel the causes and develop effective treatment of FD. In recent years; strict diagnostic criteria have evolved based on robust epidemiological data (Rome III definition of FD); symptoms should have been present for the last 3 months, with onset at least 6 months prior to diagnosis.

There is strong emerging support for the concept that the intestinal microbiome plays a critical role in mucosal inflammation. As mucosal inflammation appears to be central for symptoms in FD it appears critical to take the microbiome into consideration. It is also believed that micro-biota present in the stomach and intestines or other surfaces of biological subjects can have a significant impact on digestive or other processes and general health.

Data demonstrates that the stool microbiome poorly correlates with colonisation of the mucosa. Thus it is essential to study the microbiome colonising the mucosa of gut segments by taking targeted aseptic biopsies from defined segments of the gut rather than relying on luminal or stool samples.

It is known to acquire samples from within biological subjects, in a procedure commonly referred to as a biopsy. This is used for example to assist in performing clinical diagnosis, or the like. In order to achieve this, it is typical to insert an endoscope into the subject, with a tip of the endoscope being positioned adjacent a site to be sampled. Once in place, an instrument, such as forceps or a blade, can be inserted through a working channel of the endoscope, allowing the sample to be collected.

However, it can be difficult to extract a sample from the intestines without this being contaminated by bacteria or other substances from elsewhere in the body. For example, in order to retrieve a sample from the duodenum, it is necessary for the endoscope to pass through a subject's mouth and stomach, before reaching the duodenum. During this insertion process, parts of the working channel become contaminated with bacteria and other substances from the mouth and stomach. As a result, when the instrument is inserted into the working channel, and when samples are removed therethrough, there is the potential for these to come into contact with contaminants from the stomach, meaning it can be difficult to obtain samples from a subject without these becoming contaminated.

Thus, with currently available methods it is impossible to obtain sterile or aseptic biopsies from defined segments of the small bowel or colon. Biopsy channels of endoscopes are used to aspirate luminal content or take mucosal biopsies but working channels of endoscopes are always contaminated by luminal contents.

Given that accuracy of obtained results is heavily dependent on the quality of an obtained sample, it is important that such sampling processes are improved. This is particularly important when analysing the bacterial content of different portions of the intestines as little is currently known regarding expected micro-biota distribution, particularly in the various parts of the gastrointestinal tract.

U.S. Pat. No. 7,878,983 describes a method and device for removing tissue from a patient during a biopsy procedure utilizing a biopsy collection device. The biopsy collection device includes a body portion configured for attachment to an endoscope and a receptacle portion configured to retain a portion of tissue removed from a target site during the biopsy procedure. The biopsy collection device further includes a passageway configured to receive a biopsy sampling device, such as a pair of forceps, passing through the endoscope to the target site.

U.S. Pat. No. 7,311,674 describes a cap for a medical device. The cap includes a body defining a cavity configured to accommodate at least a portion of an end effector assembly of the medical device, and a tissue removal portion attached to the body and configured to remove a tissue sample disposed within the end effector assembly when the cavity is not accommodating the end effector assembly. Embodiments of the invention also may include a medical kit including a medical device and a cap and a method of performing a medical procedure with the cap.

### SUMMARY OF THE PRESENT INVENTION

In a first broad form the present invention seeks to provide a sampling apparatus for extracting a sample from a biological subject, the apparatus including:
a) a sampling device including a sampling tool for extracting a sample from the biological subject through a working channel of an endoscope; and,
b) an isolating member for isolating at least the sample from the working channel during extraction of the sample.

Typically the sampling device includes:
a) an elongate device body for insertion into the working channel of the endoscope; and,
b) a sampling tool provided at a distal end of the device body.

Typically the isolating member is for isolating at least the sample from an internal surface of the working channel when the sample is being extracted from the biological subject.

Typically the isolating member includes a sheath extending over at least the sampling tool.

Typically the sheath is sealed at a distal end during insertion of the sampling device into the working channel, and wherein in use, the sheath is unsealed prior to collection of the sample.

Typically the sheath is unsealed using the sampling tool.

Typically the sheath is at least one of sealed and covered using at least one of a removable cap and a plug.

Typically the sheath is sealed at least in part using a pierceable membrane.

Typically a proximal end of the sheath extends to an entry port of the endoscope, and wherein, in use, the sample is extracted from the working channel through the sheath.

Typically a proximal end of the sheath is coupled to the device body, and wherein, in use, the sample is extracted from the working channel within the sheath.

Typically the sheath includes a catheter.

Typically the isolating member includes a catheter having a proximal opening for receiving the sampling device and a distal opening sealed by at least one of a plug and a pierceable membrane.

Typically the isolating member includes first and second bodies pivotally mounted to the sampling device, the bodies being movable between an open position for use in collecting a sample, and a closed position in which the bodies define an internal cavity for containing a sample during extraction of the sample through the working channel.

Typically an actuator is used for moving the bodies between the open and closed positions.

Typically the bodies are biased towards the open position, and wherein the bodies are retained in the closed position by engagement with an internal surface of the working channel.

Typically the isolating member includes a membrane.

Typically the sampling device includes an actuator at a proximal end of the device body, the actuator being coupled to the sampling tool to allow the sample to be collected.

Typically the sampling tool includes at least one of
a) forceps;
b) a lasso;
c) a laser;
d) a blade; and,
e) an aspiration tube.

In a second broad form the present invention seeks to provide a method for extracting a sample from a biological subject, the method including:
a) positioning an endoscope in a biological subject;
b) providing a sampling apparatus within a working channel of the endoscope, the sampling apparatus including:
   i) a sampling device including a sampling tool;
   ii) an isolating member; and,
c) extracting a sample from the biological subject through a working channel of the endoscope using the sampling tool, wherein the isolating member isolates at least the sample from the working channel during extraction of the sample.

Typically the method includes:
a) positioning the sampling tool near a site to be sampled;
b) breaching the isolating member; and,
c) collecting the sample using the sampling tool.

Typically the method includes breaching the isolating member using the sampling tool.

Typically the sampling device includes an elongate device body for insertion into the working channel of the endoscope and a sampling tool provided at a distal end of the device body, wherein the isolating member includes a sheath extending over at least the sampling tool and wherein the method includes:
a) inserting the sheath and device body into the working channel;
b) breaching the sheath using the sampling tool;
c) obtaining the sample; and,
d) extracting the sample from the working channel.

Typically the method includes extracting the sample from the working channel within the sheath.

Typically the method includes extracting the sample from the working channel through the sheath.

Typically the sheath includes a catheter.

In a third broad form the present invention provides an isolating member for isolating a sampling device from a surrounding environment during extraction of sample, wherein the isolating member includes a catheter having a proximal opening for receiving the sampling device and a distal opening sealed by at least one of a plug and a pierceable membrane.

In a fourth broad form the present invention, provides a sampling apparatus for extracting a sample from a biological subject, the apparatus including:
a) a sampling device including a sampling tool for extracting a sample from the biological subject; and,
b) an isolating member for isolating at least the sample from a surrounding environment during extraction of the sample.

In a fifth broad form the present invention provides a method for extracting a sample from a biological subject, the method including:
a) positioning a sampling apparatus within a subject, the the sampling apparatus including:
   i) a sampling device including a sampling tool;
   ii) an isolating member; and,
b) extracting a sample from the biological subject using the sampling tool, wherein the isolating member isolates at least the sample from the surrounding environment during extraction of the sample.

### BRIEF DESCRIPTION OF THE DRAWINGS

An example of the present invention will now be described with reference to the accompanying drawings, in which:

FIG. 1A is a schematic diagram of an example of an endoscope;

FIG. 1B is a schematic diagram of the endoscope of FIG. 1A showing a working channel;

FIG. 1C is a schematic perspective view of a tip of the endoscope of FIG. 1A;

FIG. 1D is a schematic cross sectional view of the tip of FIG. 1C;

FIGS. 2A and 2B are schematic diagrams of first and second examples of a sampling apparatus;

FIG. 3A to 3D are schematic diagrams of an example of the process of extracting a sample from a biological subject using the sampling apparatus of FIG. 2A;

FIGS. 4A to 4D are schematic diagrams of an example of the process of extracting a sample from a biological subject using the sampling apparatus of FIG. 2B;

FIG. 5 is a schematic diagram of a third example of a sampling apparatus; and,

FIGS. 6A and 6B are schematic diagrams of a fourth example of a sampling apparatus.

FIGS. 7A and 7B are schematic diagrams of a fifth example of a sampling apparatus; and,

FIG. 8 is a graph of an example of the microbial community profiled sampled using the sampling apparatus of FIGS. 7A and 7B as opposed to a traditional sampling apparatus.

### DETAILED DESCRIPTION OF THE PREFERRED EMBODIMENTS

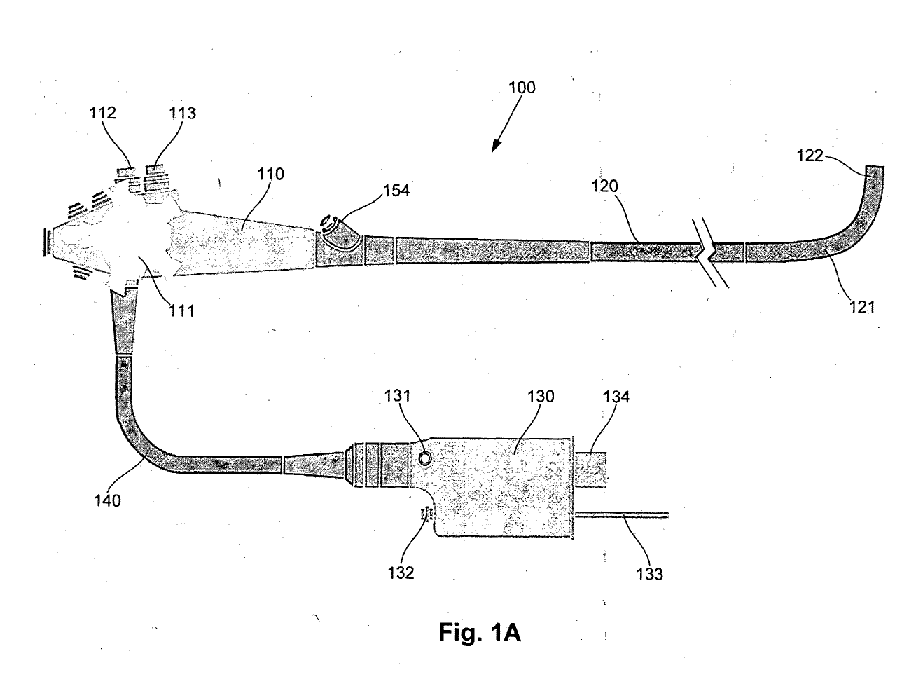

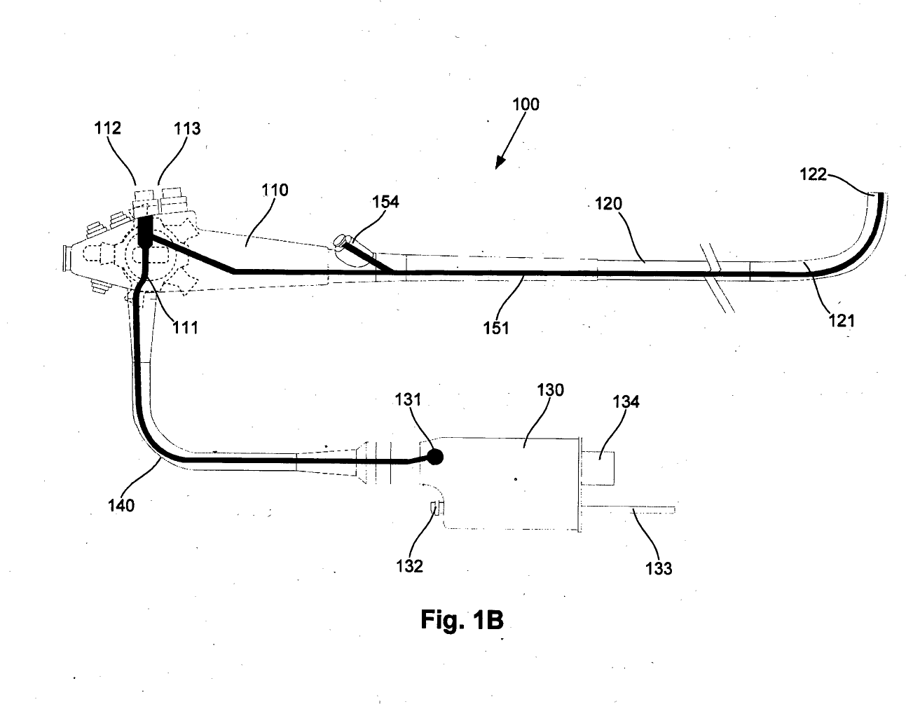

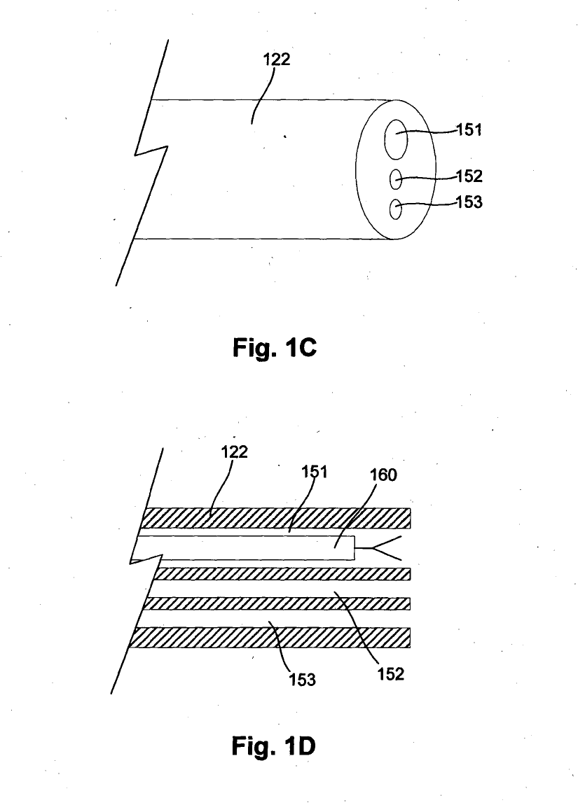

An example of an endoscope will now be described with reference to FIGS. 1A to 1D.

The endoscope 100 typically includes a control head 110, coupled to an insertion tube 120, which has a bending section 121 and ends in a tip 122. The control head 110 is further coupled to a connector assembly 130, sometimes referred to as a light guide connector. The connector assembly 130 includes a number of ports 131, 132, 133, 134 for connection to external equipment, such as a light source and imaging equipment, as well as sources of air, water and suction.

The connector assembly 130 is typically coupled to the control head 110 via an umbilical cable 140 which provides necessary connections between the ports 131, 132, 133, 134 and the endoscope 100. In particular, the endoscope typically includes a number of channels extending from the connector assembly 130, via the umbilical cable 140, to the control head 110 and from the control head 110 along the insertion tube 120 to the tip 122. These channels typically include a working channel 151, shown in FIG. 1B, which is coupled to the suction port 131 to provide suction, and which includes a biopsy port 154 to allow for insertion of instruments 160, such as forceps, or the like. The channels typically also include air/water channels 152 and an imaging/light guide 153. It will be appreciated that although, three channels only are shown, this is for the purpose of illustration and in practice any appropriate number of channels may be provided.

The control head 110 includes a number of controls to allow an operator to control operation of the endoscope 100. The controls typically include one or more articulation mechanisms 111, allowing the shape of the bending section 121 to be adjusted so that the endoscope can be inserted into the subject. Additionally, controls 112, 113 are provided for controlling the application of air and water via the air/water channel 152 and for controlling the application of suction via the working channel 151.

It will be appreciated that the above-described endoscope is therefore a typical endoscope arrangement and its operation and construction will not therefore be described in any further detail. It will also be appreciated from the following description that the techniques described herein can be implemented using any suitable endoscope, and the above described arrangement is for the purpose of illustration only.

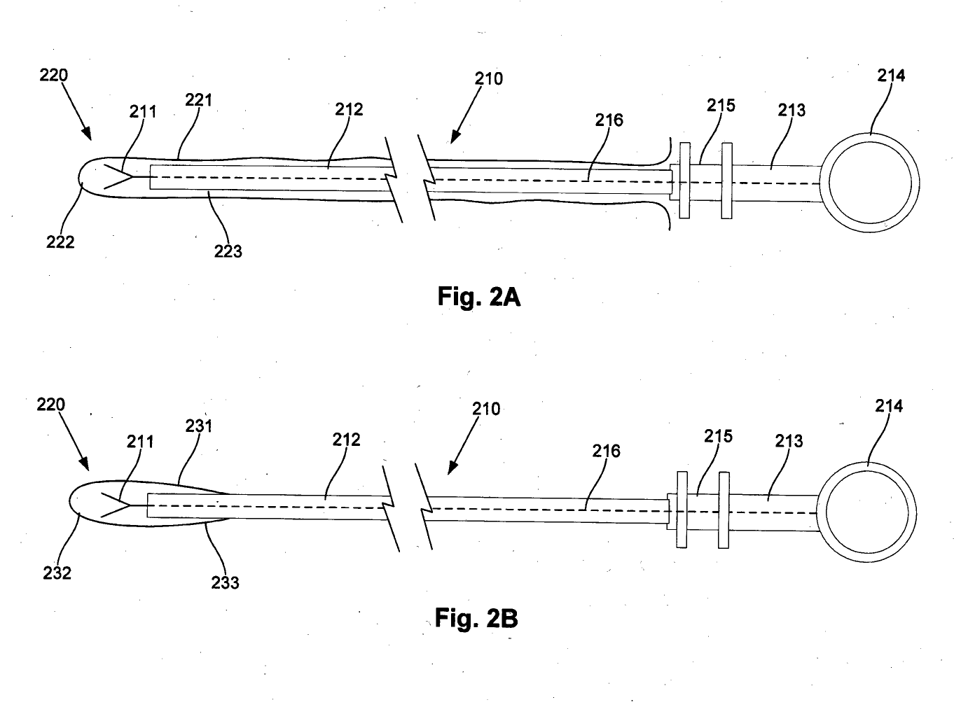

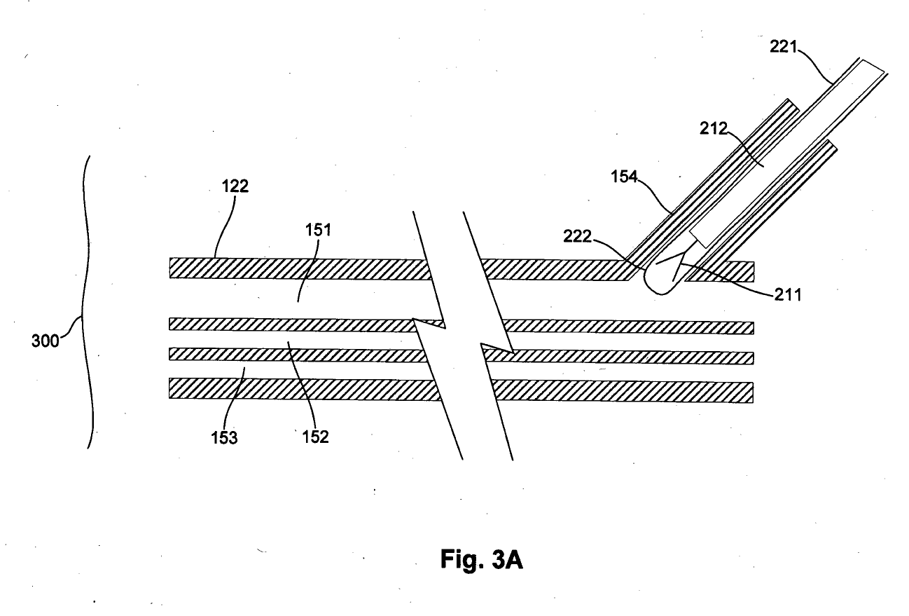

Examples of sampling apparatus for extracting a sample from a biological subject will now be described with reference to FIGS. 2A and 2B.

In this example, the sampling apparatus 200 includes a sampling device 210 which typically includes a sampling tool 211 for extracting a sample from a biological subject through the working channel 151 of an endoscope 100. The sampling apparatus further includes an isolating member 220 for isolating at least the sample from the working channel during extraction of the sample.

The isolating member 220 acts as a barrier helping to prevent the sample from coming into contact with bacteria or other contaminants within the working channel of the endoscope, thereby allowing uncontaminated samples to be removed from a subject. In particular, this allows a medical practitioner to extract a sample from a biological subject by positioning the endoscope 100 in the subject, for example, near a sampling site. Once positioned, the sampling apparatus can be introduced into the working channel, allowing a sample to be extracted from the subject through the working channel with the isolating member isolating at least the sample from the working channel during extraction of the sample.

In this regard, during insertion of the endoscope 100 into a subject, it is important that the tip 122 is open to the subject, for example to allow for the application of water, air or suction. As a result, the working channel 151 of the endoscope will tend to accumulate material from regions of the body through which the endoscope passes. For example, when positioning the endoscope in the duodenum, it is typical for the working channel to contain material from the stomach. Accordingly, by providing a sampling apparatus including an isolating member, this allows the sample to be extracted from the subject via the working channel 151, without the sample coming into contact with any contaminants therein.

In one particular example, the isolating member 220 is provided over the sampling tool, so that the sampling tool is also isolated from the working channel during insertion into the working channel, thereby further reducing the likelihood of contamination, as will be described in more detail below. In this example, once the sampling device 210 and isolating member 220 are in position, the isolating member is breached, to allow the sampling tool 211 to be used to collect a sample. This may be achieved in any suitable manner and will typically depend on the nature of the isolating member 220 and the sampling tool 211, as will be described in more detail below.

The above described arrangement therefore allows samples to be collected without contamination, whist allowing a standard endoscope to be used in the normal way. This technique therefore allows uncontaminated samples to be collected whilst using standard equipment, and without requiring medical practitioners to alter existing sampling procedures, as will become apparent from the following description.

A number of further features will now be described in more detail.

In the examples of FIGS. 2A and 2B, the sampling device 210 includes a flexible elongate device body 212 for insertion into the working channel 151 of the endoscope 100, with the sampling tool 211 being provided at a distal end of the device body. In the current example, the sampling tool 211 is a pair of forceps allowing for collection and retrieval of a sample. However, alternative tools may be used such as a knife, scalpel, lasso, laser, aspiration tube, or the like and this is not intended to be limiting.

The sampling device 210 may also include an actuator provided at a proximal end of the device body to allow the sampling tool 211 to be actuated. The nature of the actuator will depend on the preferred implementation and the nature of the sampling tool. In the current example, the actuator includes a handle 213 having a moveable thumb ring 214 and finger grips 215, allowing the thumb ring 214 to be moved relative to the handle 213. The thumb ring is attached to the forceps 211 via one or more connecting cables 216, such that movement of the thumb ring 214 can be used to allow the forceps 211 to be opened or closed. It will be appreciated from the above that the sampling device can be any form of sampling device, and that the use of a pair of forceps is for the purpose of example only.

The isolating member 220 may be of any suitable form, but in one example includes a sheath or sleeve extending over at least the sampling tool 211. However, other forms of isolating members, such as membranes, caps, plugs and enclosing bodies could be used, as will be described in more detail below.

In the example of FIG. 2A, the sheath 221 extends substantially along the length of the device body 212 and includes a sealed distal end 222 for receiving the sampling tool 211, and an open proximal end 223, allowing the sampling device to be inserted therein.

In contrast, in the example of FIG. 2B, the isolating member 220 includes a sheath 231 that extends only part way along the device body 211. Again, a distal end 232 of the sheath is sealed and sits over the sampling device 211, whilst a proximal end 233 of the sheath is attached to the sampling device body 212, rearwardly of the sampling tool 211.

The sheaths must be capable of being inserted through the working channel and into a subject, meaning they must be flexible, yet sufficiently resilient for insertion, whilst also being biologically inert and providing a barrier through which contaminants cannot cross.

In one example, the sheaths are made of a high density polyethylene, a mixture of high density polyethylene and low density polyethylene, a mixture of low density polyethylene and polypropylene, or the like. In one particular example, the sheaths are formed at least in part from resin materials or a mixture selected from, polytetrafluoroethylene (PTFE), tetrafluoroethylene-perfluoroalkoxy ethylene resin (PFA), tetrafluoroethylene-hexafluoropropylene resin (FEP), polyethylene, polypropylene, polyethylene-terephthalate, ethylene-vinyl acetate copolymer, polyolefin, polyamide, vinyl chloride, latex, natural rubber and others.

Furthermore, the sheath can be in the form of layer of material, or may alternatively include an underlying structure, coated with material. For example, the sheath could include a body having a wire coiled to define a hollow cylindrical body, which is then coated, in a manner similar to that used in the manufacture of instruments, such as forceps.

In addition, the inner and/or outer surfaces of the sheath could be coated with a material having a low coefficient of friction, such as Teflon or the like, allowing the sheath to be easily inserted into and removed from the working channel, and similarly to allow the sampling device 210 to be easily inserted into or removed therefrom.

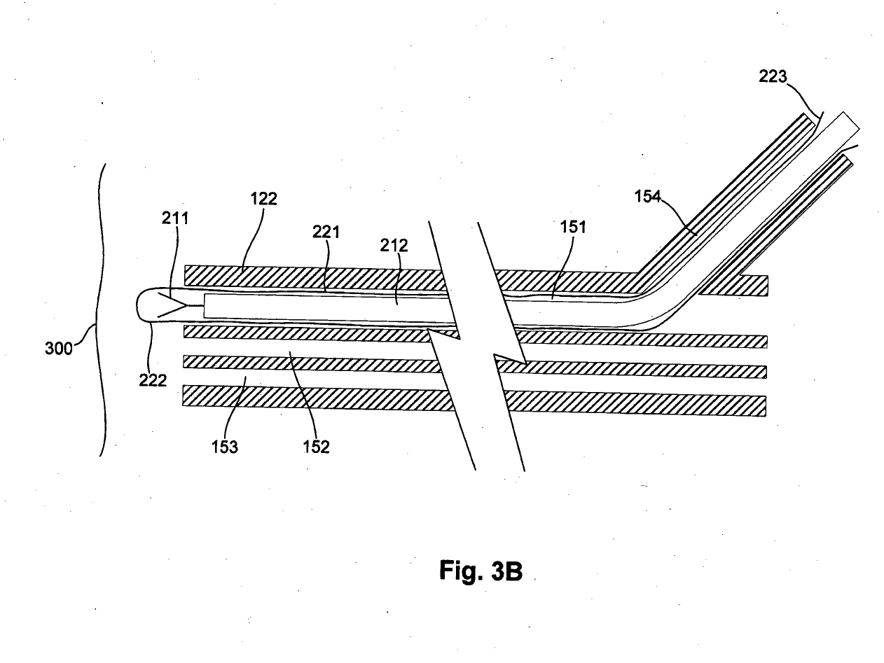

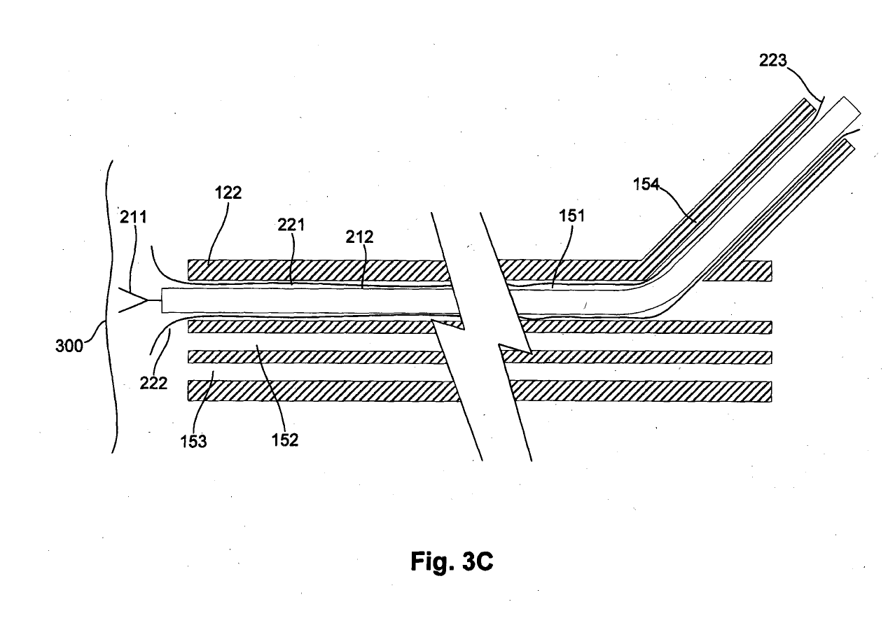

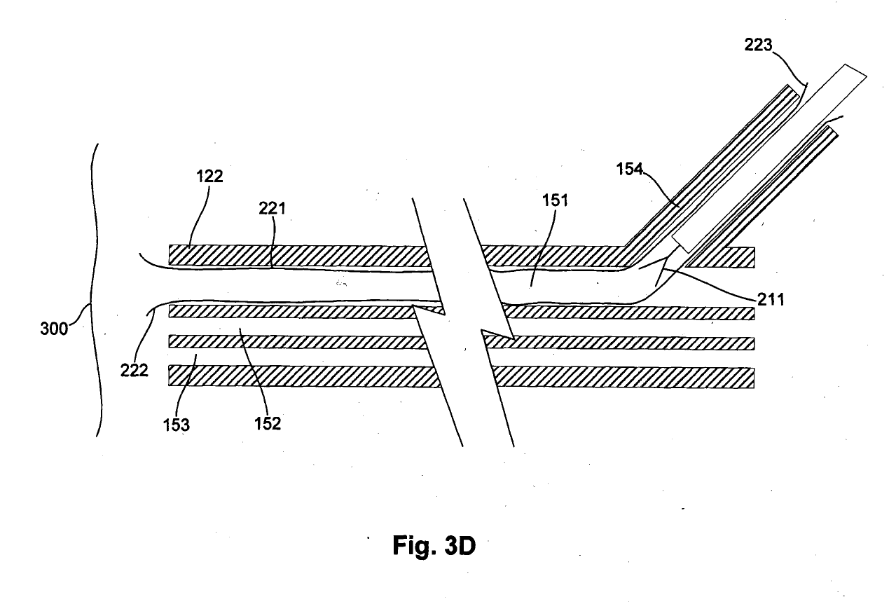

Operation of the sampling apparatus of FIG. 2A will now be described with reference to FIGS. 3A to 3D.

In this example, the sampling apparatus is initially arranged as shown in FIG. 2A with the sheath 221, extending along the elongate device body 212 as shown. The sampling device and sheath 210, 221 are inserted into the biopsy port 154 of the endoscope 100 after the endoscope 100 has been provided adjacent a site 300 to be sampled. The sampling device and sheath 210, 221 are then urged along the working channel 151, until the sampling tool 211 and the sheath distal end 222 project from the endoscope tip 122, as shown in FIG. 3B.

Positioning of the sampling apparatus may be determined using any appropriate technique. Thus, for example, the sampling device and/or sheath 221 may include markings that align with the biopsy port 154 to show when the sampling device 210 and/or sheath 220 are correctly positioned. However more typically this is achieved by having a medical professional view an image provided by the endoscope imaging system, which shows when the sampling tool 211 and sheath distal end 222 project from the endoscope tip 122.

Once the sampling device 210 and isolating member 220 are in position, the distal end of the sheath 221 is breached, to allow the sampling tool 211 to be used to collect a sample. This may be achieved in any suitable manner and will typically depend on the nature of the isolating member 220 and the sampling tool 211.

For example, the distal end 222 of the sheath 221 may include a piercable membrane provided at the distal end, so that the sampling tool 211 can be used to pierce the sheath distal end 222. This may involve utilising the actuator in order to actuate the sampling tool 211, for example by opening the jaws of forceps to rip the sheath, or alternatively may simply involve inserting the sampling device 210 further into the sheath 221, thereby urging the sampling tool 211 through the distal end 221. In order to achieve this, the medical practitioner may grasp the proximal end 222 of the sheath to thereby retain the sheath in position whilst the sampling device is urged through the distal end 221. Alternatively, the proximal end 222 may include a fitting, such as a resilient ring, or the like, which engages with the biopsy port 154 and prevents the sheath being inserted further into the working channel 151.

However, alternative mechanisms for breaching the isolating member can be used. For example, the sheath 221 could incorporate an actuation mechanism that operates when the sheath is in position, for example through manual or automated mechanisms.

In any event, once the distal end 222 of the sheath 221 has been breached, the sampling device 210 may be used to collect a sample using standard biopsy techniques, and this will not therefore be described in any further detail.

Once this has been completed, the sampling device 210 can be extracted through the sheath 221, whilst the sheath remains in place, as shown in FIG. 3D. Once the sampling device 210, and in particular the collected sample, have been removed the sheath 221 can then be removed from the working channel 151. Alternatively, further instruments may be inserted into the sheath 221, allowing further work to be performed as required.

Accordingly, in the above described example, the sampling tool 211 is covered with an isolating member 220 as the sampling tool is inserted through the working channel 151. Consequently the sampling tool 211 remains isolated from the contents of the working channel 151 thereby preventing contamination of the sampling tool 211.

In one particular example, the isolating member ensures that the sampling tool cannot come into contact with an internal surface of the working channel 151 thereby ensuring any material on the internal surface cannot contaminate the sampling tool. Furthermore, once the sample has been collected, the sampling tool and sample can be withdrawn through the sheath. As a result, the sample is also isolated from the working channel as the sample is withdrawn.

Thus, in the above described example, the sampling tool 211 and removed sample are protected from contaminants within the working channel. This allows samples to be collected from internally within a biological subject without contamination by substances from other parts of the body.

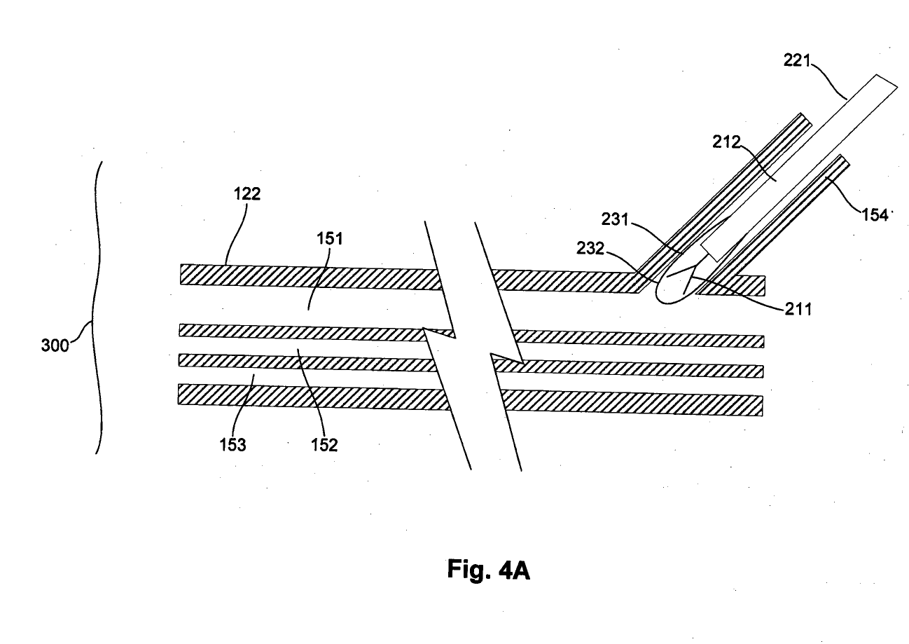

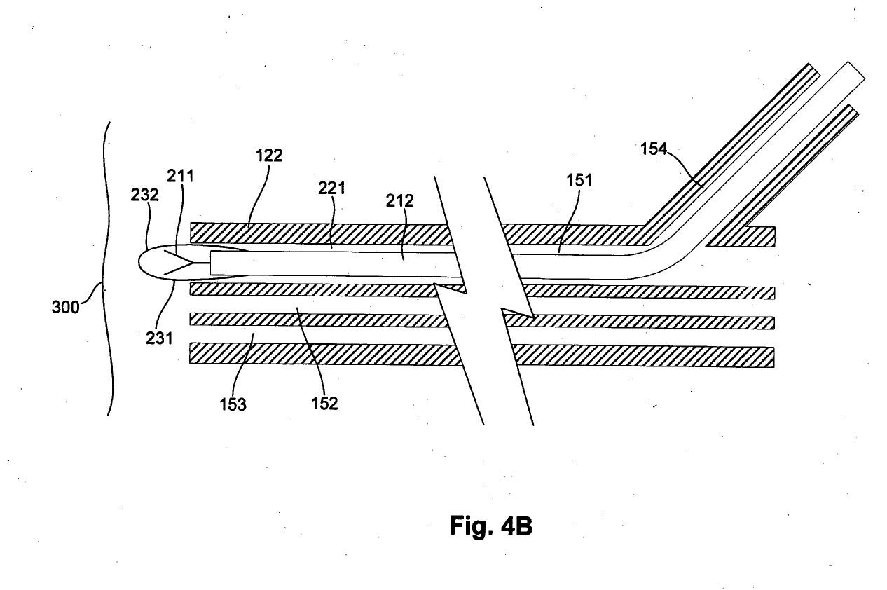

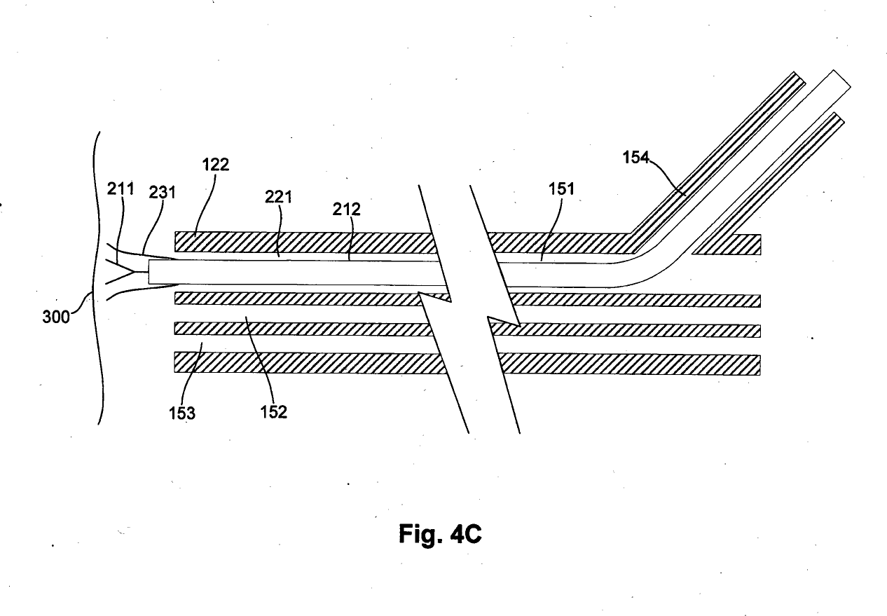

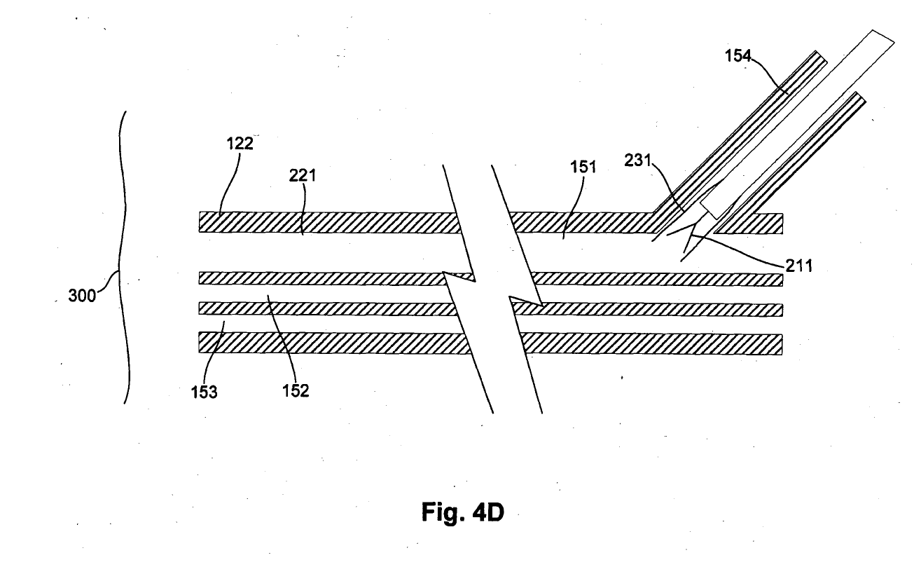

Operation of the sampling apparatus of FIG. 2B will now be described with reference to FIGS. 4A to 4D.

In this example, the sampling device. 210 is again inserted into the working channel with the sheath 231 sealed at the distal end 232, as shown in FIGS. 4A and 4B. In this example, once the sampling device 210 is correctly positioned, the actuator is actuated causing the sampling tool 211 to pierce the sheath 231 thereby allowing the sampling tool 211 to collect a sample in the normal way.

In this instance, as the sampling device 210 is withdrawn into the working channel, the sheath 231 is dragged back into the working channel 151 with the sample. In this instance, the sheath 231 is pulled back around the sample as the sampling tool 211 enters the working channel 151, thereby ensuring the sample remains isolated at least from the working channel 151 of the endoscope.

Accordingly, it will be appreciated that the second example of FIG. 2B provides similar functionality to the example of FIG. 2A, albeit with the sheath being removed from the working channel 151 as part of the process of removing the sampling device.

It will be appreciated from this, that a wide variety of different configurations can be used to provide the required isolation between the sample and the working channel.

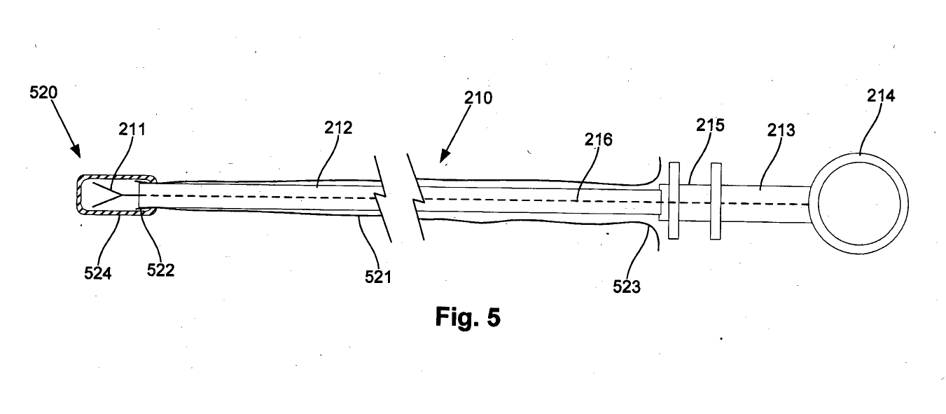

In a further variation shown in FIG. 5, the distal end of the sheath is sealed using a removable cap, although a plug or other similar arrangement could be used.

In this example, the isolation member includes a sheath 521 similar to that described above with respect to FIG. 2A, having distal and proximal ends 522, 523. The distal end 522 is sealed by a cap 524, which is provided over the sampling tool 211 and attaches to the sampling device body 212. In this instance, once the sampling device 210 has been inserted into the working channel 151, the cap 524 can be removed, for example through actuation of the sampling tool 211, or urging the sampling tool against the cap, allowing the sampling apparatus to function in substantially the same manner as the example of FIG. 2A.

In this scenario, it is typical for the cap to be made of a degradable or biologically inert material, allowing the cap to remain within the subject, with the cap being expelled from the digestive system, in due course.

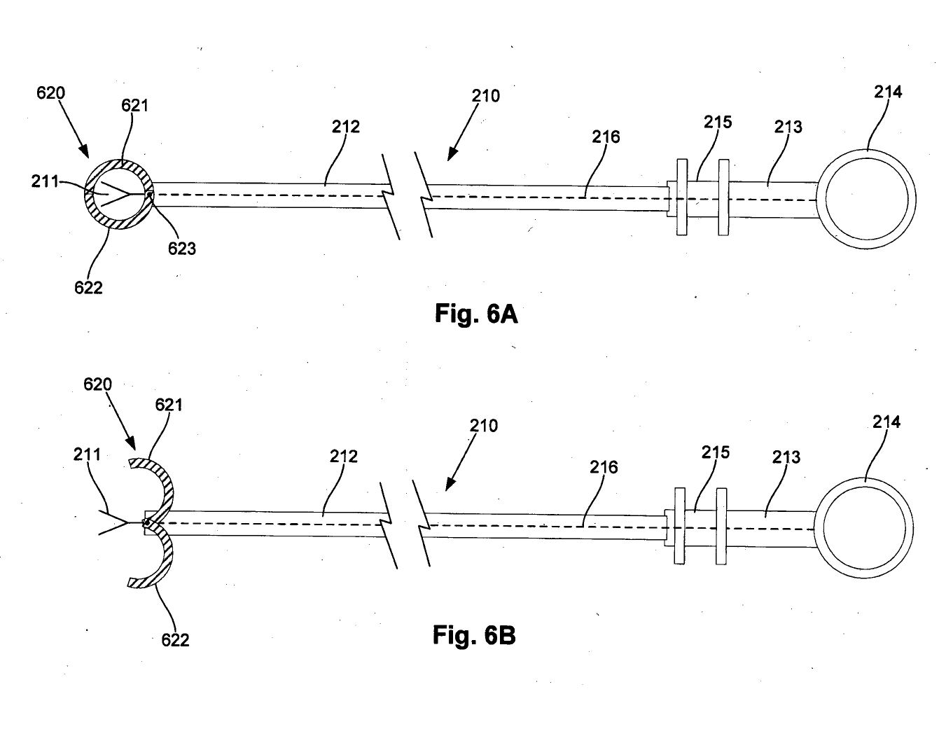

A further variation is shown in FIGS. 6A and 6B.

In this example, the sampling apparatus includes an isolation member 620 including two substantially hemispherical bodies 621, 622, pivotally coupled to the sampling device 210 via a pivotal connection 623. In use, the bodies 621, 622, are movable between an open position for use in collecting a sample, and a closed position in which the bodies define an internal cavity for containing a sample during extraction of the sample through the working channel, as shown in FIGS. 6A and 6B respectively.

In one example, in the closed position, the bodies 621, 622 encompass the sampling tool 211 so that the sampling tool is isolated from the external environment, and in particular, the working channel 151. By contrast, in the open position, the sampling tool is exposed, allowing a sample to be collected in the normal way. Accordingly, in use, the bodies can be provided in the closed arrangement during insertion of the sampling device into the working channel 151, and during removal of a collected sample, whilst the bodies can be moved into an open position when sample collection is being performed.

In one example, movement of the bodies between the open and closed positions can be controlled using an actuator, similar to the actuator used for controlling the forceps. In one particular example, the bodies 521, 522 can act as the sampling tool, allowing the forceps jaws to be replaced with the bodies 521, 522.

However, alternatively, the bodies can be biased towards the open arrangement, using a biasing mechanism such as a spring or the like. In this instance, the bodies are sized to cooperate with the working channel, so that the bodies are retained in the closed position within the working channel 151 by engagement with an inner surface of the working channel. This avoids the need to provide a separate actuator, and ensures that the bodies are closed when the sampling tool 211 is in the working channel.

In a further example, the bodies 521, 522 can actually act as the sampling tool, allowing the sample to be collected whilst isolating the collected sample from the working channel 151.

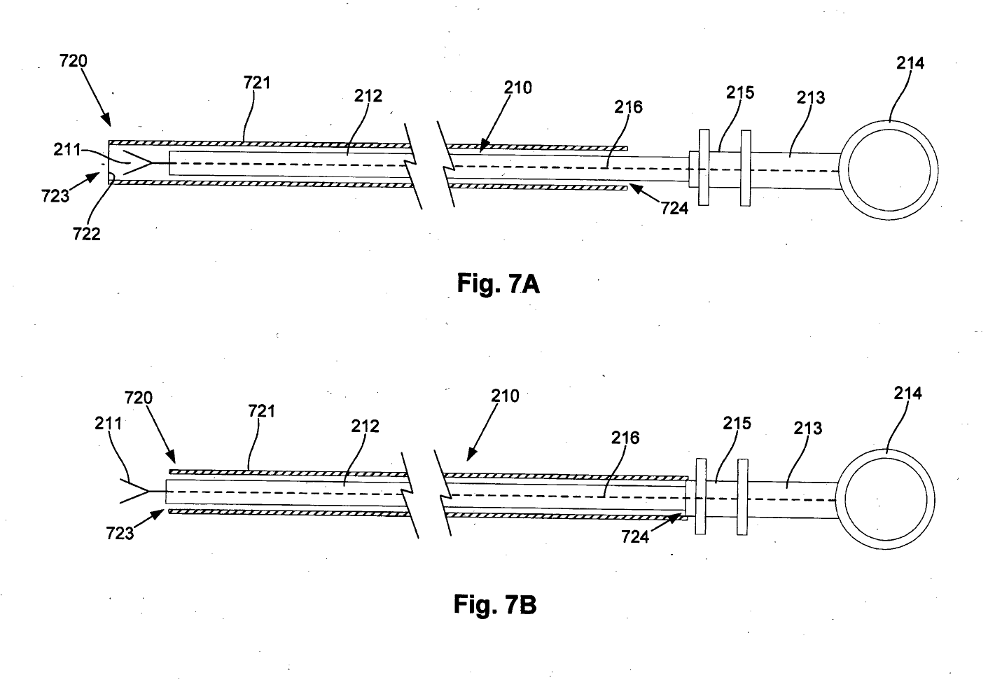

In a further example, the isolation member includes a sheath in the form of a catheter having a proximal end opening for receiving the sampling device and a distal end opening sealed by a pierceable membrane. An example of this arrangement will now be described with reference to FIGS. 7A and 7B.

In this example, the sampling apparatus includes an isolation member 720 including a catheter 721 sealed at a distal end opening 723, either by a pierceable membrane 722, or another sealing member such as a plug or the like. In use, the sampling device 210 is inserted into the catheter 721 at the proximal end opening 724, so that the sampling tool 211 is contained within the catheter 721 proximate to the membrane 722, as shown in FIG. 7A. The catheter 721 can then be inserted into the working channel 151 of the endoscope 100, when the endoscope 100 is positioned within the subject, until the distal end of the catheter 721 projects from the working channel 151 and into the subject.

The sampling device 210 can then be urged through the catheter 721 so that the sampling tool 211 pierces the membrane 722, as shown in FIG. 7B, thereby allowing sample collection to be performed. Following this, the sampling device 210 can be withdrawn into the catheter 721, which is then removed from the working channel 151, in a manner similar to that described with respect to previous examples, thereby allowing the sample to be extracted substantially without contamination.

Accordingly, in this example, the isolation member is formed from a catheter 721 having one end sealed by a pierceable membrane, which is pierced by forceps or another sampling tool.

In another example, the isolation member and sampling device could be introduced into the body of a subject using mechanisms other than an endoscope. For example, an incision could be used to introduce the catheter 721 directly into a region of a subject's body. The sampling device 210 can then be urged along the catheter towards the distal end, thereby piercing the membrane 722, allowing a sample to be collected. The sampling device can then be extracted from the subject through the catheter 721, with the catheter 721 then being removed as required.

It will therefore be appreciated that whilst the use of an endoscope is highly desirable in allowing samples to be easily collected from a range of locations, this is not intended to be limiting, and that in practice, the apparatus could be used to isolate the sampling device from a surrounding environment, such as a subject's body, without requiring use of an endoscope.

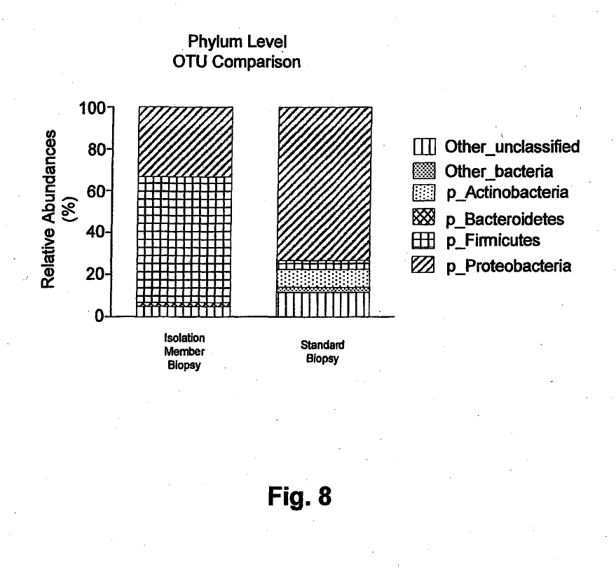

To demonstrate the effectiveness of the isolation members, duodenal biopsies were collected from a subject using an isolation member, and using standard biopsy forceps. A graph showing a comparison of the microbial community profile as sampled using the isolation member arrangement and a standard biopsy arrangement are shown in FIG. 8. These preliminary data reveal significant differences between the microbial community profile of samples taken from the same site with a normal biopsy or the isolation member, highlighting that the use of the isolation members reduces contamination of biopsy samples, in turn allowing a more accurate determination of microbial profile. This will allow for enhanced study of FD and other related conditions, in turn allowing enhanced treatment programs to be determined.

In any event, it will be appreciated from the above that isolation members can be used to ensure that samples removed from the subject are uncontaminated by substances, such as bacteria, that have entered the working channel during insertion of the endoscope into the subject. This provides a mechanism for collecting uncontaminated samples, which can be particularly important in diagnosing conditions associated with operation of the gastro-intestinal tract.

It will also be appreciated that the isolation member may be of any appropriate form, including, but not limited to sheaths, sleeves, membranes, caps, plugs, catheters or enclosing bodies. Furthermore, it will be appreciated that the isolation member can be incorporated into the sampling device, or could alternatively be a separate device that functions with a separate sampling device. This latter arrangement is particularly advantageous as this allows existing sampling devices to be used in the above described sampling methods. It will therefore be appreciated that the term isolation member can encompass any arrangement, including one or more components, that is capable of isolating a sampling device from a surrounding environment, such as a working channel of an endoscope, and that reference to specific arrangements is not intended to be limiting.

The term sheath could encompass any arrangement that extends generally over and along a body of a sampling device, including a catheter, and that the term is not intended to be limited to a rigid or non-rigid body.

The terms proximal and distal are used to refer to opposing ends of a device, with general reference to their position relative to an operator in use. However, this is merely for the purpose of illustration and their use is not intended to be limiting.

Persons skilled in the art will appreciate that numerous variations and modifications will become apparent. All such variations and modifications which become apparent to persons skilled in the art, should be considered to fall within the spirit and scope that the invention broadly appearing before described.

What is claimed is:

1. A sampling apparatus for extracting a sample from a biological subject, the apparatus including:
a) a sampling device including a sampling tool for extracting a sample from the biological subject through a working channel of an endoscope; and,
b) an isolating member for isolating at least the sample from the working channel during extraction of the sample.

2. A sampling apparatus according to claim 1, wherein the sampling device includes:
a) an elongate device body for insertion into the working channel of the endoscope; and,
b) a sampling tool provided at a distal end of the device body.

3. A sampling apparatus according to claim 1, wherein the isolating member is for isolating at least the sample from an internal surface of the working channel when the sample is being extracted from the biological subject.

4. Apparatus according to claim 1, wherein the isolating member includes a sheath extending over at least the sampling tool.

5. Apparatus according to claim 4, wherein the sheath is sealed at a distal end during insertion of the sampling device into the working channel, and wherein in use, the sheath is unsealed prior to collection of the sample.

6. Apparatus according to claim 5, wherein the sheath is unsealed using the sampling tool.

7. Apparatus according to claim 4, wherein the sheath is at least one of sealed and covered using at least one of a removable cap and a plug.

8. Apparatus according to claim 4, wherein the sheath is sealed at least in part using a pierceable membrane.

9. Apparatus according to claim 4, wherein a proximal end of the sheath extends to an entry port of the endoscope, and wherein, in use, the sample is extracted from the working channel through the sheath.

10. Apparatus according to claim 4, wherein a proximal end of the sheath is coupled to the device body, and wherein, in use, the sample is extracted from the working channel within the sheath.

11. Apparatus according to claim 4, wherein the sheath includes a catheter.

12. Apparatus according to claim 1, wherein the isolating member includes a catheter having a proximal opening for receiving the sampling device and a distal opening sealed by at least one of a plug and a pierceable membrane.

13. Apparatus according to claim 1, wherein the isolating member includes first and second bodies pivotally mounted to the sampling device, the bodies being movable between an open position for use in collecting a sample, and a closed position in which the bodies define an internal cavity for containing a sample during extraction of the sample through the working channel.

14. Apparatus according to claim 13, wherein an actuator is used for moving the bodies between the open and closed positions.

15. Apparatus according to claim 13, wherein the bodies are biased towards the open position, and wherein the bodies are retained in the closed position by engagement with an internal surface of the working channel.

16. Apparatus according to claim 1, wherein the isolating member includes a membrane.

17. Apparatus according to claim 1, wherein the sampling device includes an actuator at a proximal end of the device body, the actuator being coupled to the sampling tool to allow the sample to be collected.

18. Apparatus according to claim 1, wherein the sampling tool includes at least one of:
a) forceps;
b) a lasso;
c) a laser;
d) a blade; and,
e) an aspiration tube.

19. A method for extracting a sample from a biological subject, the method including:
a) positioning an endoscope in a biological subject;
b) providing a sampling apparatus within a working channel of the endoscope, the sampling apparatus including:
i) a sampling device including a sampling tool;
ii) an isolating member; and,
c) extracting a sample from the biological subject through a working channel of the endoscope using the sampling tool, wherein the isolating member isolates at least the sample from the working channel during extraction of the sample.

20. A method according to claim 19, wherein the method includes:
a) positioning the sampling tool near a site to be sampled;
b) breaching the isolating member; and,
c) collecting the sample using the sampling tool.

21. A method according to claim 20, wherein the method includes breaching the isolating member using the sampling tool.

22. A method according to claim 19, wherein the sampling device includes an elongate device body for insertion into the working channel of the endoscope and a sampling tool provided at a distal end of the device body, wherein the isolating member includes a sheath extending over at least the sampling tool and wherein the method includes:
a) inserting the sheath and device body into the working channel;
b) breaching the sheath using the sampling tool;
c) obtaining the sample; and,
d) extracting the sample from the working channel.

23. A method according to claim 22, wherein the method includes extracting the sample from the working channel within the sheath.

24. A method according to claim 22, wherein the method includes extracting the sample from the working channel through the sheath.

25. A method according to claim 22, wherein the sheath includes a catheter.

26. An isolating member for isolating a sampling device from a surrounding environment during extraction of sample, wherein the isolating member includes a catheter having a proximal opening for receiving the sampling device and a distal opening sealed by at least one of a plug and a pierceable membrane.

27. A sampling apparatus for extracting a sample from a biological subject, the apparatus including:
a) a sampling device including a sampling tool for extracting a sample from the biological subject; and,
b) an isolating member for isolating at least the sample from a surrounding environment during extraction of the sample.

28. A method for extracting a sample from a biological subject, the method, including:
a) positioning a sampling apparatus within a subject, the sampling apparatus including:
i) a sampling device including a sampling tool;
ii) an isolating member; and,
b) extracting a sample from the biological subject using the sampling tool, wherein the isolating member isolates at least the sample from the surrounding environment during extraction of the sample.
